(19) United States
(12) Patent Application Publication
(10) Pub. No.: US 2011/0301518 A1
(43) Pub. Date: Dec. 8, 2011

(54) Orthopedic cast-embeddable window device

(76) Inventor: Chris E. Wiggins, Pascagoula, MS (US)

(21) Appl. No.: 12/802,466

(22) Filed: Jun. 8, 2010

(51) Int. Cl. A61F 5/00

(52) U.S. Cl. 602/8

(57) ABSTRACT

An orthopedic cast window is provided for creating an access to a site of injury, such as a broken limb. The device comprising: A cast-embeddable hollow body has a tubular member defining an opening and having a first peripheral edge and a second peripheral edge. The opening is sized to extend about the site of injury without obstructing access to the injured site. An inner flange is secured to the first peripheral edge, extending transversely to the tubular member; an outer flange secured to the second peripheral edge, extending transversely to the tubular member. When embedded in the cast, the inner flange and at least a part of the tubular member become embedded in the cast bandages. A window cover member is configured to be detachably positioned within the opening in the tubular member in a covering relationship to said opening to allow easy access to the site of injury.

### BACKGROUND OF THE INVENTION

This invention relates to the treatment of living tissues and/or cells by providing a limited access to a site of injury in the living tissue or cell. More particularly, the invention relates to a window device that is incorporated in an orthopedic cast during formation of the cast.

Orthopedic casts have been in use for over a century to treat extremity injuries, mainly fractures. Conventional orthopedic casting methods for compound fractures do not allow for a medically sound environment for a wound to heal in that wounds are not allowed to breathe when covered completely by an orthopedic cast. A health service provider therefore is often faced with the decision of either concentrating on the wound or the fracture. By concentrating solely on the wound, the fractured bone will not set properly. On the other hand, by concentrating first on the bone and ignoring the wound exposes the patient to an increased risk of infection.

Often times, a physician needs an access to the site of injury to either deliver a treatment, such as for instance electromagnetic treatment, or to observe the wounded tissue and its response to the treatment, to provide a drain of accumulated fluid and the like Typically, a hole is cut or punched in the orthopedic cast via a vibrating cast saw or the cast is removed to gain access to the injury site.

Removal or sawing of the orthopedic cast is technically demanding for a health service provider in that special care must necessarily be taken in order to avoid injury to the patient, such as cast saw burns, or to prevent disturbing the bone cells that have not yet properly fused. In addition, removal of the orthopedic cast by sawing or punching is very stressful to the patient since it may cause pain and skin irritation associated with the mechanical breaking of the set cast. Furthermore, there are hygienic concerns involved with the current cast cutout removal methods such as soiling of the cast edges from wound drainage or medication.

The present invention contemplates elimination of the drawbacks associated with conventional techniques and provision of an embeddable window insert that can be positioned in the cast and provide easy access to a site of injury whenever required by a physician.

### SUMMARY OF THE INVENTION

It is therefore an object of the present invention to provide an embeddable window device for an orthopedic cast.

It is another object of the invention to provide a window device that allows easy access to a site of injury for administration of therapeutic treatment or observation.

These and other objects of the invention are achieved through a provision of an orthopedic cast window device adapted for creating an access to a site of injury. The window device comprises a hollow body having a tubular member defining an opening and having a first peripheral edge and a second peripheral edge, said opening being configured to extend about the site of injury. An inner flange is fixedly secured to the first peripheral edge of the tubular member and extends transversely to the tubular member; an outer flange is affixed to the second peripheral edge and extends transversely to the tubular member. The inner flange and the tubular member become embedded in cast bandages when the hollow body is positioned over the site of injury.

A removable cover member is provided for covering the opening and allowing access to the site of injury upon demand.

### BRIEF DESCRIPTION OF THE DRAWINGS

Reference will now be made to the drawings, wherein like parts are designated by like numerals, and wherein

FIG. 1 is a perspective exploded view of the orthopedic cast window insert according to the present invention.

FIG. 2 is a perspective view of an alternative embodiment of the window device of the present invention.

FIG. 3 is a perspective view of the orthopedic cast window insert of the present invention embedded into an orthopedic cast.

FIG. 4 illustrates a step of embedding the window insert of the present invention into a short leg cast.

FIG. 5 illustrates a step of positioning a window cover into the hollow body of the window device.

FIG. 6 illustrates a step of completing a short leg cast using the window device of the present invention.

### DETAIL DESCRIPTION OF THE INVENTION

Turning now to the drawings in more detail, numeral 10 designates an orthopedic window device in accordance with one embodiment of the present invention. The window device comprises a hollow body 12 and a window cover 14. The hollow body 12 comprises a tubular member 16 having an interior surface 18 and an exterior surface 20. The tubular member 16 can be rectangular, cylindrical, oval, etc., depending on the desired applications.

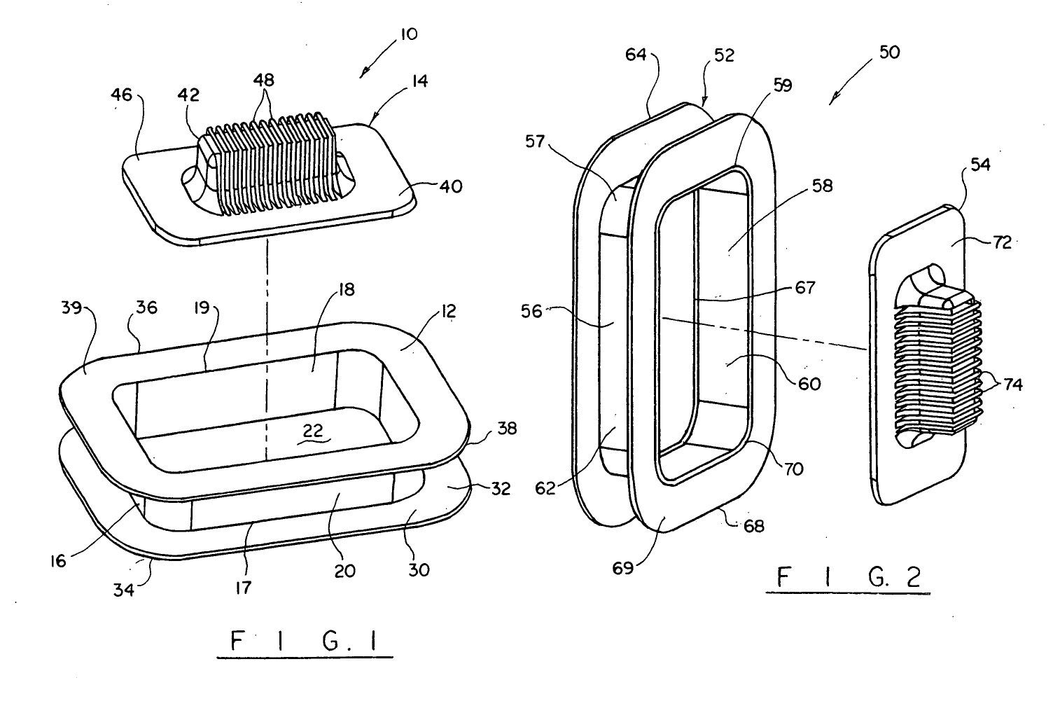

The tubular member 16 defines an opening 22, which is configured to extend in a surrounding relationship about a site of injury, such as a broken extremity. In the embodiment of FIG. 1, the interior surface 18 forms a relatively smooth wall defining the opening 22.

An inner flange 30 is unitary secured to a peripheral edge 17 of the tubular member 16, extending outwardly from the tubular member 16 in a transverse relationship to the peripheral edge 17. The inner flange 30 has an outer surface 32 and an inner, contact surface 34. An outer flange 36 is unitary secured to the opposite peripheral edge 19 of the tubular member 16, extending outwardly from the tubular member 16 in a transverse relationship to the peripheral edge 19. The outer flange 36 has an inner surface 38 and an exterior surface 39. In one aspect of the invention, the flanges 30 and 36 are substantially mirror images of each other and extend in a parallel relationship to each other.

The lateral dimensions of the peripheral inner flanges 30 are sufficient to be engaged by cast bandages during embedment of the window device 10 into a cast. The lateral dimensions of the outer flange 36 are sufficient to partially overlay the cast bandages, as will be described in more detail hereinafter.

The window cover 14 comprises a substantially planar body 40 sized and shaped to fit into the opening 22 of the hollow member 12. In one aspect of the invention, the planar body 40 is configured to frictionally engage the interior surface 18 of the tubular body 16 when the cover 14 is placed in the opening 22.

A raised gripping member 42 is secured to an upper surface 46 of the planar body 40. The gripping member 42 is configured to be grasped by a user's hand when positioning or removing the cover 14 from the hollow body 12. The gripping member 42 may be shaped as a rectangular member, an oval member or in any other convenient configuration. The gripping member 42 may be provided with friction-enhancing outer surface or with ridges 48 to facilitate gripping by the user. The outward extension of the gripping member 42 from the planar body 40 is selected to be at slightly smaller than the width of the tubular member 16, so that the gripping member does not extend from the opening 22 beyond the plane of the exterior surface 39 of the outer peripheral flange 36 when the window cover is engaged with the hollow body 12.

Turning now to the embodiment of FIG. 2, the window device is generally designated by numeral 50. The window device 50 comprises a hollow body 52 and a window cover 54. The hollow body 52 comprises a tubular member 56, which defines a through opening 58. The tubular member 56 has a relatively smooth inner surface 60 and a relatively smooth outer surface 62.

An inner peripheral flange 64 is unitary formed with the tubular member 56 and secured to the inner peripheral edge 57 of the tubular member 56. The inner flange 64 extends in a transverse relationship to the tubular member 56. A part 65 of the inner flange 64 extends outwardly from the tubular body 56, while a second part 67 of the inner flange 64 extends inwardly to the inner edge 57. The second part 67 of the inner flange 56 forms a ledge or a stop member for positioning of the window cover 54 when the window cover 54 is engaged with the hollow body 52.

An outer peripheral flange 68 is unitary formed with the tubular member 56 and secured to an outer peripheral edge 59 of the tubular member 56. The outer flange 68 extends in a transverse relationship to the tubular member 56. A part 69 of the outer flange 68 extends outwardly from the tubular body 56, while a second part 70 of the outer flange 68 extends inwardly in relation to the outer edge 59 and the opening 58. The second part 70 of the outer flange 68 forms a ledge or a stop member for outward displacement of the window cover 54 when the window cover 54 is engaged with the hollow body 52.

The window cover 54 is similar to the window cover 14 inasmuch as it comprises a planar body 72 and a raised gripping member 74. The planar body is sized and shaped to fit into the opening 58. The planar body 72 is sized at least slightly smaller than the opening defined by the inwardly extending parts 67 and 70 of the inner flange 64 and the outer flange 68, respectively. The inwardly extending part 67 prevents the window cover 54 from contacting the skin of a patient, while the inwardly extending part 70 prevents disengagement of the window cover 54 from its position within the opening 58.

The window devices 10 and 50 are formed from a pliable, soft, resilient material that retains its shape when positioned in a cast opening. The window devices 10 and 50 can be manufactured by vacuum molding, injection molding and various other manufacturing processes. It is preferable that the window devices 10 and 50 be packaged in sterile fashion since they will be placed adjacent to a wound, and this would also allow them to be employed in operating room settings.

In operation, a health service provider treats the wound site in a conventional manner and places a soft flexible stocking 80 on a wounded extremity, such as a leg 82 of a patient. The technician then cuts an opening in the stocking 80 corresponding to the size of an area of interest. The window device 10 or 50 is then placed on the leg 82, such the openings 22 (or 58) surround the area of interest. The technician is then wraps plaster bandages 84 around the broken limb 82 making sure than the inner flange 30 (or 64) is in contact with the stocking 80.

Conventional plaster bandages consist of a cotton bandage that has been impregnated with Plaster of Paris, which hardens after it has been made wet. Plaster of Paris is calcined Gypsum, ground to a fine powder by milling. When water is added, the more soluble form of calcium sulfate returns to the relatively insoluble form and the limb, which has been wrapped in the plaster bandages, is immobilized.

The limb immobilization can be achieved using synthetic materials, such as fiberglass bandages impregnated with polyurethane, or thermoplastic bandages. Regardless of the plaster material used, the window device 10 or 50 can be used in providing access to the wounded site.

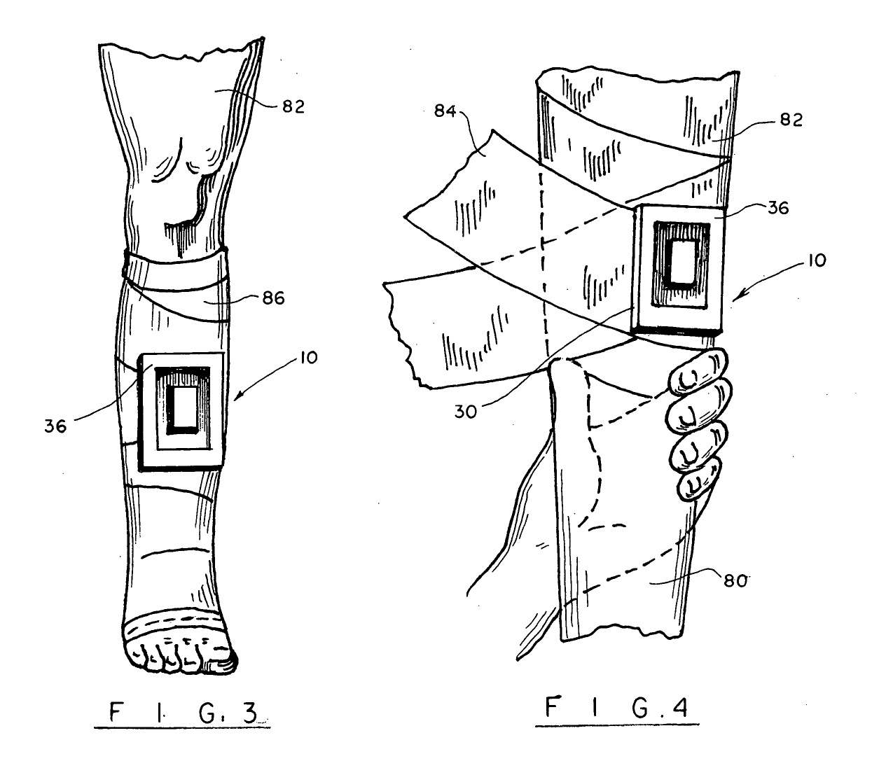

As the bandages 84 are applied, the technician makes sure that the bandages overlap the inner flange 30 (or 64), engaging the outer surface of the inner flange 30 (64). The tubular members 16, 56 prevent the bandages from extending over the openings 22, 58 and covering the wounded area. See FIGS. 3-6.

FIG. 3 illustrates a short leg cast 86 and the window device 10 embedded in the cast. The outer flange 36 overlaps a portion of the cast 86, contacting the bandages with its inner surface. The inner flange 30 and almost entire tubular member 16 are covered with the cast bandages 84. Once the window device is secured by the bandages 84, the technician continues wrapping the bandages 84 to complete the short leg cast 86, as shown in FIG. 6.

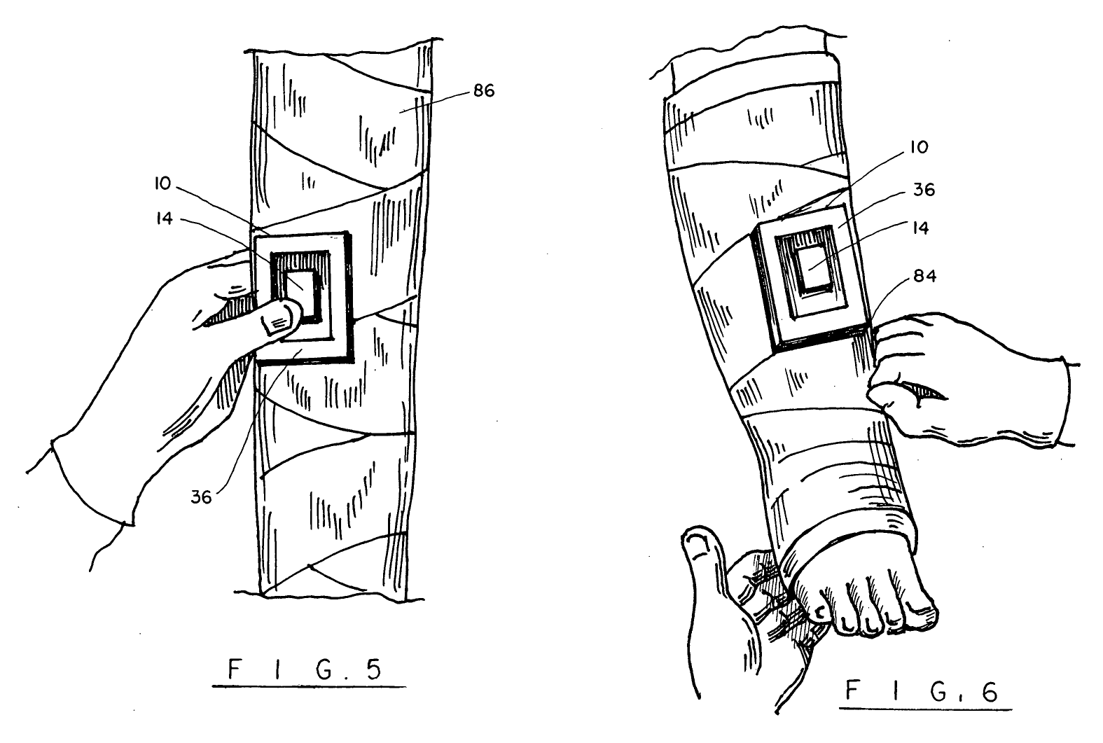

The technician then positions the window cover 14 (or 54) in the openings 22 (or 58) formed in the tubular members 16 (or 56). A slight pressure may be applied to the window cover 14 (54), as shown in FIG. 5 to make sure that the gripping member 42 (74) does not extend from the cast 86 beyond the plane of the outer peripheral flange 36 (68). The cast is allowed to harden. If desired, a flexible wrap layer can be placed over the formed cast and the embedded window device so as to protect the window device from damage and the injury—from contamination.

When a physician needs to administer a therapeutic treatment or examine the injured site, the physician grips the gripping member 42 (74) pulling it out of the opening 22 (58). The tissue of the limb can be seen through the opening in the hollow body 12 (52). Any required treatment can be then administered to the site of injury without having to cut or punch a window in the cast 86.

When the window cover is in place it protects the wound from external contamination and prevents “window” edema, the bulging of tissues outward at the site of the cast window. Repeated access through the window device is easily done without ever requiring the use of cast saw. The cast edges and padding remain unsoiled. t is envisioned that the window device 10 (50) can be manufactured in a variety of sizes to accommodate small or large wounds, arm or leg locations.

Many changes and modifications can be made in the present invention without departing from the spirit thereof. I, therefore, pray that my rights to the present invention be limited only by the scope of the appended claims.

I claim:

1. An orthopedic cast window device adapted for creating an access to a site of injury, the device comprising:
a cast-embeddable hollow body having a tubular member defining an opening and having a first peripheral edge and a second peripheral edge, said opening being configured to extend about the site of injury;
an inner flange secured to the first peripheral edge and extending transversely to the tubular member;
an outer flange secured to the second peripheral edge and extending transversely to the tubular member; and
a window cover member configured to be detachably positioned within the opening in the tubular member in a covering relationship to said opening.

2. The device of claim 1, wherein said inner flange and said tubular member are configured for at least partial embedment in an orthopedic cast.

3. The device of claim 1, wherein the outer flange is configured to overlay an orthopedic cast.

4. The device of claim 1, wherein the window cover member comprises a generally planar body and a raised gripping member secured to the planar body, said gripping member facilitating positioning and removal of the window cover member in relation to the hollow body.

5. The device of claim 4, wherein the planar body is sized at slightly smaller than the opening in the tubular member.

6. The device of claim 4, wherein said tubular member comprises an inner surface, and wherein the planar member is configured to frictionally engage the inner surface when positioned in the tubular member.

7. The device of claim 1, wherein said inner flange comprises an inwardly-extending part extending into the opening of the tubular body, said inwardly extending part limits movement of the window cover member into the opening.

8. The device of claim 1, wherein said outer flange comprises an inwardly-extending part extending into the opening of the tubular body, said inwardly extending part of the outer flange preventing inadvertent release of the window cover member from said opening.

9. The device of claim 1, wherein said hollow body is formed from a flexible moldable material.

10. An orthopedic cast window device adapted for creating an access to a site of injury, the device comprising:
a hollow body having a tubular member defining an opening and having a first peripheral edge and a second peripheral edge, said opening being configured to extend about the site of injury;
an inner flange secured to the first peripheral edge and extending transversely to the tubular member;
an outer flange secured to the second peripheral edge and extending transversely to the tubular member; and
a window cover member configured to be detachably positioned within the opening in the tubular member in a covering relationship to said opening, wherein said inner flange and said tubular member are configured for at least partial embedment in an orthopedic cast.

11. The device of claim 10, further comprising a means for limiting movement of the window cover member within said opening.

12. The device of claim 11, wherein said movement limiting means comprises an inwardly-extending part formed on said inner flange, said inwardly-extending part of the inner flange limiting movement of the window cover member into the opening.

13. The device of claim 11, wherein said movement limiting means comprises an inwardly-extending part formed on said outer flange, said inwardly-extending part of the outer preventing inadvertent release of the window cover member from said opening.

14. The device of claim 10, wherein the outer flange is configured to overlay an orthopedic cast.

15. The device of claim 10, wherein the window cover member comprises a generally planar body and a raised gripping member secured to the planar body, said gripping member facilitating positioning and removal of the window cover member in relation to the hollow body.

16. The device of claim 15, wherein the planar body is sized at slightly smaller than the opening in the tubular member.

17. The device of claim 15, wherein said tubular member comprises an inner surface, and wherein the planar member is configured to frictionally engage the inner surface when positioned in the tubular member.

18. A method of positioning an orthopedic cast over a limb, comprising the steps:
providing a flexible hollow body having a tubular member defining an opening and having a first peripheral edge and a second peripheral edge, an inner flange secured to the first peripheral edge and extending transversely to the tubular member, an outer flange secured to the second peripheral edge and extending transversely to the tubular member;
positioning a protective stocking over an injured limb;
forming an aperture in the stocking, said aperture corresponding to a size and location of a site of injury;
positioning the hollow body over said stocking, while aligning the opening of the tubular body with the aperture and the site of injury;
wrapping cast bandages over the stocking, while forming a cast over the injured limb, and while engaging the inner flange of the hollow body and embedding the inner flange and at least a part of the tubular body within the cast;
providing a window cover member and detachably positioning the window cover member within the opening in the tubular member in a covering relationship to said opening.

19. The method of claim 18, wherein the step of wrapping cast bandages comprising a step of positioning the outer flange in an overlapping relationship over at least a portion of the cast.

20. The method of claim 18, further comprising s step of providing a means for limiting movement of the window cover member within said opening.

21. The method of claim 20, wherein said movement limiting means comprises an inwardly-extending part formed on said inner flange, said inwardly-extending part of the inner flange limiting movement of the window cover member into the opening.

22. The method of claim 20, wherein said movement limiting means comprises an inwardly-extending part formed on said outer flange, said inwardly-extending part of the outer preventing inadvertent release of the window cover member from said opening.

23. The method of claim 18, further comprising a step of removing the window cover member and gaining access to the site of injury upon demand.
